(19) United States
(12) Patent Application Publication
(10) Pub. No.: US 2026/0241322 A1
(43) Pub. Date: Aug. 20, 2026

(54) SUBTERRANEAN ODOR REMEDIATION SYSTEM

(71) Applicant: Ricky Allan Jacobs, Carrollton, GA (US)

(72) Inventor: Ricky Allan Jacobs, Carrollton, GA (US)

(21) Appl. No.: 19/056,663

(22) Filed: Feb. 18, 2025

(51) Int. Cl. B01D 53/04; A61L 9/014; A61L 9/16

(52) U.S. Cl. CPC B01D 53/0446; A61L 9/014; A61L 9/16; A61L 2209/13; A61L 2209/22; B01D 2257/90; B01D 2258/02

(57) ABSTRACT

An subterranean wastewater odor remediation system a subterranean container adjacent an air release valve connected to a sewer main. A conduit connects to the air release valve to communicate gas emitted therefrom to an inlet disposed within the subterranean container. An odor control unit having a manifold is in communication with the inlet. A gas treatment media surrounds the manifold and fills a portion of the container and covers the manifold while leaving an upper zone free of the gas treatment media. An air admittance valve is disposed in the upper zone and connects to a pipe extending downward through the gas treatment media toward a bottom of the container. A container lid sealably connects to a top of the subterranean container. A vent is installed in the container lid to exchange air from above surface with air and gas reaching the upper zone and thereby removing odor from gas.

### FIELD OF INVENTION

This invention relates generally to the treatment of wastwater gases. More particularly, the invention relates to a subterranean odor remediation system.

### BACKGROUND OF INVENTION

Odor control in wastewater treatment is a crucial aspect of environmental management and public health. Wastewater contains organic and inorganic compounds that, when broken down by microbial activity, release volatile gases responsible for foul odors. Effective odor control is essential for maintaining compliance with environmental regulations, improving worker safety, and ensuring community well-being.

Odors in wastewater treatment typically arise from anaerobic decomposition of organic matter which produces hydrogen sulfide (H₂S), ammonia (NH₃), and volatile organic compounds (VOCs). Collection systems and sewer networks are associated with stagnant wastewater and industrial discharges contribute to odor formation. Treatment processes include primary clarifiers, sludge digestion, and aeration tanks which can release odorous gases. In addition, biosolids handling such as dewatering, storage, and disposal of sludge generate strong odors.

Hydrogen Sulfide (H₂S) is a well known Rotten egg smell, is highly toxic at high concentrations. Ammonia (NH₃) is pungent, irritant to the respiratory system. Volatile Organic Compounds (VOCs) include mercaptans, skatole, and organic acids responsible for a range of odors. Sulfur Compounds such as methyl mercaptan and dimethyl sulfide, are common in anaerobic digestion.

Proper aeration is required to reduce anaerobic conditions. If possible, pH control is used to minimize the release of odorous gases. Location control is also used to regulating industrial discharges.

Chemical treatment concern employs oxidizing agents (e.g., chlorine, hydrogen peroxide) which neutralize odor-causing compounds. Iron salts (e.g., ferric chloride, ferrous sulfate) precipitate sulfides and reduce odor. Biological additives can be used to promote microbial degradation of odorous compounds.

Physical and mechanical controls such as activated carbon adsorption or creative traps reduce odor molecules in filtration systems. Scrubbers (chemical or bio-filters) remove gases using chemical reactions or biological activity.

Sealed covers and ventilation systems have been employed to reduce emissions from open tanks and sludge processing areas. Biological control, such as bio-filtration uses bacteria to break down odor-causing compounds. In certain applications, composting of biosolids helps stabilize organic matter and reduce odor.

In main sewer lines there exist sewer forced main pipes which pump waste waters through the pipes. There are high points in the pipes which collect corrosive gases such hydrogen sulfide and methane. In these areas, air release valves (ARV) are installed to vent these gases above surface. Unfortunately, the nearby residents are subject to these toxic gases and odors.

Many times, the municipalities either turn off the AVR leading to pipe corrosion or install an above ground odor control unit including having to place a concrete slab on the owner's property and use a container housing on the slab which is also unsightly.

Environmental agencies set limits on air emissions from wastewater treatment plants. Public complaints about odors can lead to stricter enforcement and the need for advanced odor management solutions. Engaging the community and implementing proactive odor monitoring systems can help maintain good relations and compliance with regulations. Existing odor remediation units include air release valve units above ground with enclosures which are quite unsightly. In addition, in some cases they are ineffective in removing odors from surrounding areas.

Odor control remains a critical challenge in wastewater treatment, requiring a combination of engineering, mechanical, chemical, and biological strategies to minimize its impact. The instant invention aims to overcome the problems associated with odor remediation.

### SUMMARY OF THE INVENTION

It is an object to improve odor remediation.

A further object is improve environment and aesthetics in the field of odor remediation of wastewater.

Still another object is to provide an improved odor remediation system.

Yet another object is to provide an subterranean wastewater gas remediation system.

Accordingly, an aspect of the invention is directed to an subterranean wastewater odor remediation system. The subterranean wastewater gas remediation system includes a subterranean container adjacent an air release valve connected to a sewer main. The subterranean container preferably has a drain system, which can include holes in a bottom thereof to permit water and fluid to pass therethrough via gravity.

A conduit, which can be flexible, connects to the air release valve in a manner to communicate gas emitted from the air release valve with an inlet pipe, such as PVC pipe, disposed within the container. The PVC pipe is connected to an odor control unit. In this regard, a manifold is operably installed in the odor control unit in communication with the inlet pipe and includes at least one, and preferably several pipes, which can be made of PVC) and at least a portion of which has one or more opening formed in the manifold pipes to release the gases. A suitable screen can be provided around the manifold opening(s) to permit gas to exit the manifold while preventing lager material from entering the manifold.

In this regard, there is a gas treatment media is provided surrounding the manifold and filling at least a portion of the subterranean container and covering the manifold leaving an upper zone free of the media in which an air admittance valve is installed and is connected to a pipe (e.g., PVC pipe) extending downward toward the bottom of the subterranean container. A container lid is provided to seal a top of the subterranean container, save for a vent which is installed therein to exchange air from above surface with the air/gas reaching the upper zone to removes odor from the gas.

### BRIEF DESCRIPTION OF THE DRAWINGS

FIG. 1 is a side schematic view illustrating the invention.

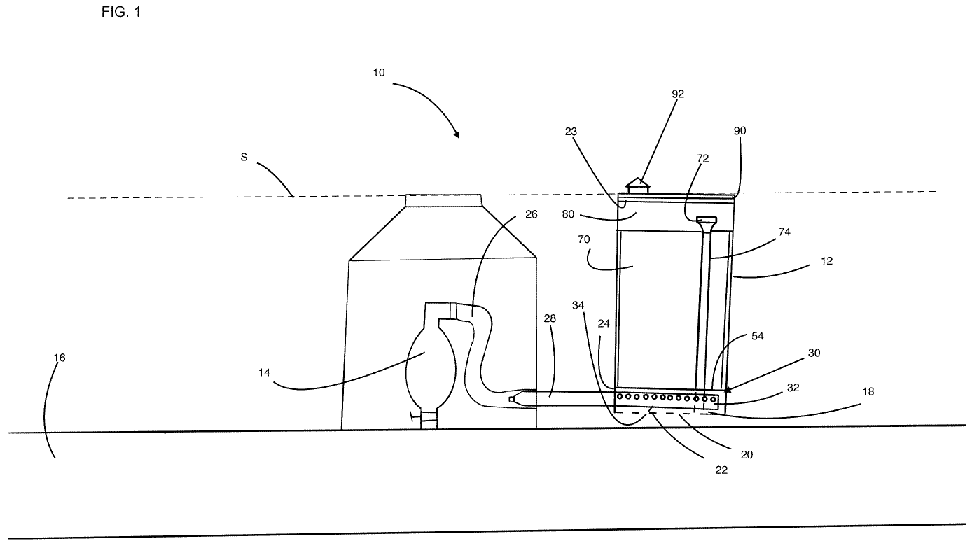

FIG. 2 is a top view illustrating a part of the invention.

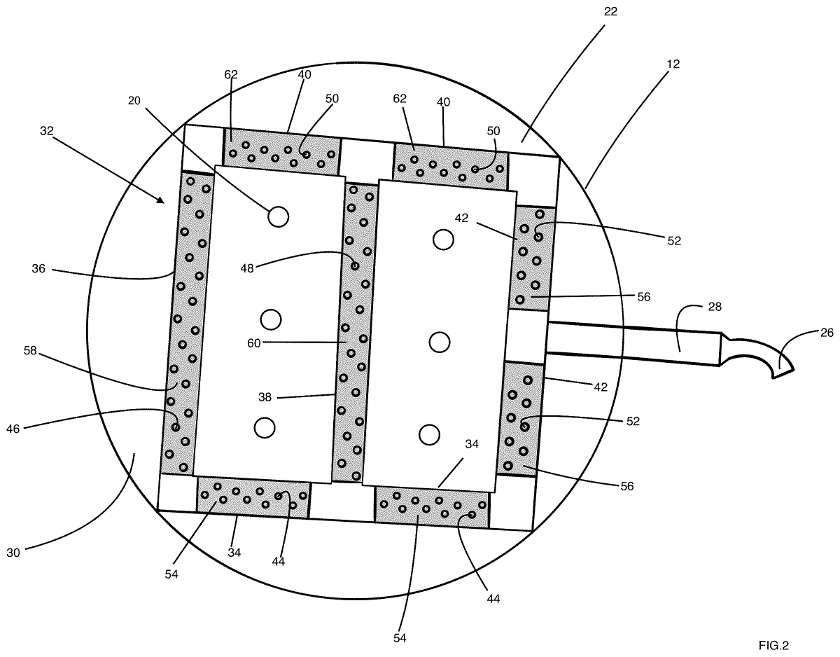

### DETAILED DESCRIPTION OF THE PREFERRED EMBODIMENT

Referring now to the drawing, the subterranean wastewater gas remediation system of the instant invention is generally designated by the numeral 10 disposed beneath surface S. The subterranean wastewater gas remediation system 10 includes a subterranean container 12 which by way of example is formed of concrete, but it is contemplated other materials can be employed. The subterranean container 12 is disposed adjacent an air release valve 14 which is connected to a sewer main 16. The subterranean container 12 preferably has a drain system 18, which can include holes 20 in a bottom 22 thereof to permit water and fluid to pass therethrough via gravity. The subterranean container 12 also has a side opening 24.

A conduit 26, which can be flexible, connects to the air release valve 14 in a manner to communicate gas emitted from the air release valve 14 with an inlet pipe 28, such as PVC pipe, which extends into the subterranean container 12 through side opening 24. The PVC pipe 28 is connected to an odor control unit In this regard, the order control unit 30 includes a manifold 32 operably installed in the odor control unit 30 in communication with the inlet pipe 28. The manifold 32 includes at least one, here shown as several interconnected pipes 34, 36, 38, 40 and 42, which can be made of PVC for example. At least a portion of each of the pipes 34, 36, 38, 40 and 42 has openings 44, 46, 48, 50 and 52 formed therein to permit release gases. The openings 44, 46, 48, 50 and 52 can be of a suitable size, for example, ½ inch, to readily permit gas to escape. A suitable screen material 54, 56, 58, 60 and 62 can be provided around the openings 44, 46, 48, 50 and 52 to permit gas to exit the manifold, while preventing lager material, namely the odor removing media 70, from entering the manifold 32.

In this regard, gas treatment media 70 of a type known in the art (granular pellet for absorbing or filtering gas) is provided surrounding the manifold 32 and filling at least a portion of the subterranean container 12 covering the manifold 32 while leaving an upper zone 80 free of the gas treatment media 70. An air admittance valve 72 is disposed in the upper zone 80 and is connected to a pipe 74 (e.g., PVC pipe) extending downward toward the bottom 22 of the subterranean container 12. A container lid 90 is provided to seal a top 23 of the subterranean container 12, save for a covered vent 92 which is installed therein to exchange air from above surface S with the air/gas reaching the upper zone 80. The system 10 thereby removes odor from the gas through movement of the gas in this system.

It will be understood that the embodiment of the present invention assembly and method that have been illustrated are merely exemplary and that a person skilled in the art can make variations to the shown embodiment without departing from the intended scope of the invention. All such variations, modifications and alternate embodiments are intended to be included within the scope of the present invention as defined by the claims.

1. A subterranean wastewater odor remediation system for an air release valve connected to a sewer main, which includes:
a subterranean container adjacent the air release valve;
a conduit connected to the air release valve in a manner to communicate gas emitted from the air release valve to an inlet disposed within said subterranean container;
an odor control unit having a manifold in communication with said inlet;
a gas treatment media surrounding said manifold and filling at least a portion of said subterranean container and covering said manifold and leaving an upper zone free of said gas treatment media;
an air admittance valve disposed in said upper zone and connected to a pipe extending downward through said gas treatment media toward a bottom of said subterranean container;
a container lid connected to a top of said subterranean container; and
a vent installed in said container lid to exchange air from above surface with air and gas reaching said upper zone from the air release valve and thereby removing odor.

2. The subterranean wastewater odor remediation system of claim 1, wherein said
manifold has a plurality of pipes and at least a portion of which have an opening formed therein to permit release the gas.

3. The subterranean wastewater odor remediation system of claim 1, wherein said pipes are made of PVC and have a plurality of openings.

4. The subterranean wastewater odor remediation system of claim 1, wherein said subterranean container includes holes in a bottom thereof to permit water and fluid to pass therethrough via gravity.

5. The subterranean wastewater odor remediation system of claim 1, wherein said inlet pipe is PVC.

6. The subterranean wastewater odor remediation system of claim 1, wherein said conduit is flexible.

7. The subterranean wastewater odor remediation system of claim 1, which includes a screen material disposed around said manifold to permit gas to exit said manifold while preventing said gas treatment media from entering said manifold.

8. The subterranean wastewater odor remediation system of claim 1, wherein said container lid is sealably connected to said subterranean container.
